(19) United States
(12) Patent Application Publication
(10) Pub. No.: US 2003/0078163 A1
(43) Pub. Date: Apr. 24, 2003

(54) Apparatus and method for application of liquids

(76) Inventor: Kenneth A. Budke II, Cedar Falls, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/241,034

(22) Filed: Sep. 10, 2002

Related U.S. Application Data
(60) Provisional application No. 60/322,392, filed on Sep. 11, 2001.

(51) Int. Cl. A61M 35/00; A46B 11/06; A01N 25/00

(52) U.S. Cl. 504/116.1; 604/1; 401/42

(57) ABSTRACT

An apparatus and method for targeted, efficient, and easy application of a fluid such as herbicide or insecticide, to a surface, such as a plant leaf or stem. The apparatus includes an elongated hand-manipulatable handle defining a fluid reservoir, a sealable fluid inlet to the reservoir, an application head, and a fluid control between application head and reservoir. The method includes storing a fluid to be applied in a portable hand-held manner, transferring the fluid to a targeted surface by capillary action of the stored fluid to an application head that allows swiping or touching of the head to the target and resists spillage or wind drift of the fluid. In one aspect of the invention, a kit includes an apparatus as described in combination with an instruction card that includes a measure or rule for mixing concentrate of a fluid for use with the apparatus.

This application is based on U.S. Provisional Application 60/322,392, filed Sep. 11, 2001.

### INCORPORATION BY REFERENCE

The entire contents of U.S. Provisional Application 60.322,392, filed Sep. 11, 2001, is incorporated by reference herein.

### I. BACKGROUND OF THE INVENTION

There is a need in the art for an economical, safe and easy to handle system of applying liquids, e.g. liquid chemicals, to a targeted surface or area. Some non-inclusive specific examples of such liquids are liquid herbicides, insecticides, or fertilizers.

In the case of liquid herbicides, a conventional method of application includes wiping, spraying or pouring herbicide onto the desired area. However, because of factors such as wind, applying herbicide in these manners is inefficient. It can result in overspray which wastes the chemical and could adversely affect non-targeted things, especially in the case of liquids that are toxic to plant or animal life.

Oftentimes, too much herbicide is administered and/or it is placed on too large of an area. In addition, these means of application are not as safe as the user easily can be exposed to sometimes hazardous herbicide.

Similar problems exist with respect to other chemicals, including but not limited to insecticides and fertilizers.

Therefore, there is a real need for an improvement in the art. There is a real need in the art for an apparatus and method which solves or improves over the problems and deficiencies in the art.

### II. BRIEF SUMMARY OF THE INVENTION

The present invention is a portable hand-held liquid application apparatus and method. The apparatus comprises a liquid reservoir, preferable sealable, in its handle; a refilling connection; a fluid control device through which to control liquid flow, and optionally its rate of flow, from the reservoir to the application head; and an application head for transferring the liquid to an external targeted surface or object.

The present invention provides for a safe and economical means of dispensing and applying liquid herbicide to a focused targeted area. With the invention, the user can accurately apply the exact amount of liquid herbicide, regardless of external factors like wind. In addition, the user can apply the herbicide without physically being exposed to the chemicals.

The invention is applicable to other fluids, including analogous liquid chemicals.

Further objects, features, aspects, and/or advantages of the invention include a method and apparatus which:

a. is economical to manufacture, has a minimum number of non-complex, low-cost parts, which can be assembled efficiently;

b. is economical to use, minimizing the amount needed by facilitating high accuracy targeting of the fluid, and by facilitating use of concentrate chemicals, mixable just before use, and loadable into the device, instead of buying more expensive pre-mixed chemical;

c. is accurate for targeted application of the fluid, deterring use of excess or misplaced application, or dripping or other loss of fluid;

d. is easy and quick to use, allowing for standing operation and freedom of movement with minimal operational tasks, minimizing bending or stooping, or operation of such things as manual pumps or spray wands;

e. is flexible is use, allowing for different types of liquids and applications, as well is being highly manipulatable to target both exposed items or surfaces, or partially exposed items or surfaces (e.g. allows reaching under or around bushes or structures;

f. is durable, reuseable, and easily maintained.

### III. BRIEF DESCRIPTION OF THE DRAWINGS

FIG. 1 is an assembly drawing of one embodiment of the present invention.

FIG. 2 is an exploded view of the embodiment of FIG. 1.

FIG. 3 is an enlarged, isolated partial view of a fluid control device that could be used in an embodiment of the present invention.

### IV. DETAILED DESCRIPTION OF EXEMPLARY EMBODIMENTS OF INVENTION

A. Overview

In order to better understand the general invention, one exemplary embodiment will now be described in detail. This example is not intended to limit the invention. It will be appreciated that the invention can take many forms and embodiments. Variations obvious to one skilled in the art will be included with the invention.

B. General Environment of Use

The exemplary embodiment is discussed in the context of a tool to apply liquid herbicide to targeted plants. This is only one example of implementation of the invention. Other types of chemicals or fluids and other tasks or implementations are also possible.

C. General Structure of Exemplary Embodiment

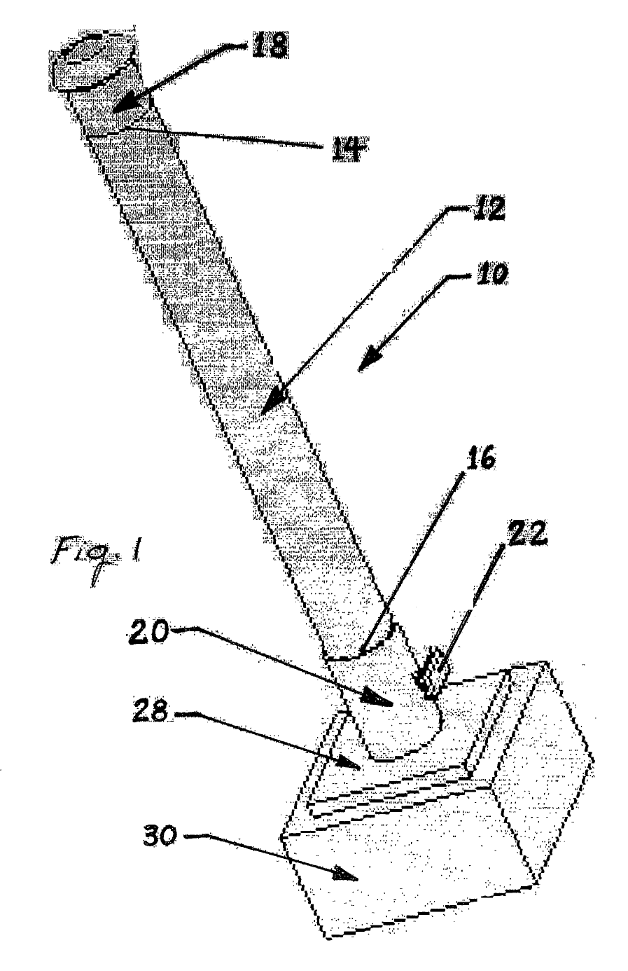

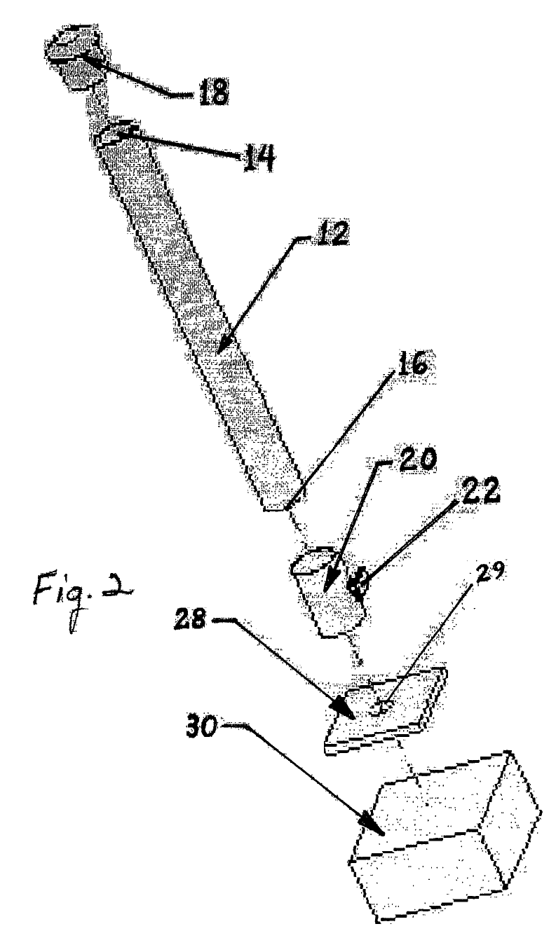

The exemplary embodiment is illustrated at FIGS. 1-3. As can be seen, this embodiment (designated generally by reference numeral 10), includes the following basic components:

(a) An elongated handle 12 defining an interior fluid reservoir, for example, a five-foot long, one-inch diameter rigid PVC tube, having opposite open ends 14 and 16.

(b) A removable, screw-on closure 18, for example a one-inch diameter rigid PVC end cap having a male externally threaded portion that screws in and out of the internally matingly threaded open female end 14 of handle 12.

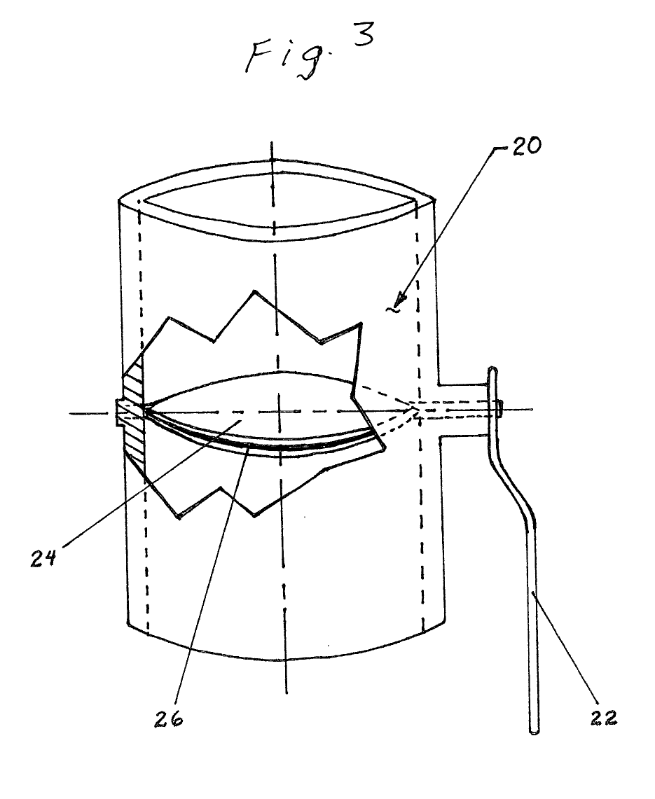

(c) A fluid control device 20, having an external hand-operated lever or knob 22, which can be manually operated between closed and open positions to either block or seal off fluid in the internal reservoir from access to open end 16 of the elongated handle 12, or permit liquid to flow to open end 16. Control device 20 could be, for example, a butterfly valve having a plastic disc 24 and a rubber seat 26 rotatable in a one-inch diameter PVC tubular section (see FIG. 3).

(d) A mounting plate 28, for example a three or four inch square, quarter-inch thick sheet of plastic with an opening 29 therethrough. Opening 29 has a diameter less than the inside diameter of the tube of valve 20. The diameter of opening 29 could be any of a number of sizes, depending upon various factors. Plate 28 can be fixed to the distal end valve 20 with opening 29 generally aligned with the bore through handle 12, e.g. by PVC cement or otherwise.

(e) An application head 30, for example a four-inch square, one-inch thick sponge attached to the mounting plate 28, e.g. with adhesive or by other methods. Sponge could be a four by four inch polyester household sponge attached by adhesive or otherwise to plate 28. This arrangement gives a bigger interface between the sponge and its attachment to the device than if attached only to the distal end of valve 20 or handle 12.

D. Operation

In operation, control valve 22 is moved to the closed position and fluid to be dispensed is loaded into the reservoir of the handle by removing cap 18 and pouring the liquid in. Cap 18 is then installed on handle 12. Device 10 is manually carried to the location of a target. Control 22 is operated so that disc 24 of valve 20 is rotated out of closed position to open a fluid pathway from the reservoir in the handle, through opening 29 in mounting plate 28 to application head 30.

In this embodiment, the sponge or sponge-like material of application head 30 wicks fluid from its reservoir side into a substantial part of head 30.

The user manually manipulates device 10 so that an outer surface of head 30 comes into contact with a targeted surface, and wipes the head 30 on the targeted surface to release the fluid onto the surface.

As is well-known with sponges, the amount of fluid that is to be deposited on the targeted surface usually can be controlled by the amount of pressure exerted on the sponge head 30 relative to the targeted surface. The more pressure, usually converts to more fluid deposited on the targeted surface.

The user manually withdraws head 30 from contact with the targeted surface. Thus, device 10 allows for quick, efficient placement and use of the chemical for its intended purpose.

E. Options and Alternatives

As previously mentioned, the above description of exemplary embodiments is given by way of example only, and not by way of limitation to the invention. As can be easily understood, this invention can take many forms and embodiments in addition to the one described immediately above. Some specific examples of options, alternatives, or variations are described below.

For example, the length of the elongated handle may be altered depending upon the preferences of the user or the requirements of the application. Similarly, the diameter of the elongated handle, closure, and fluid control device may be increased or decreased accordingly. In addition, the fluid control device may be any type of valve, such as a ball or plug valve, and its materials of construction may be altered depending upon the liquid being dispensed. For example, the valve seat may be fabricated of a synthetic polymer to resist the degradation of a corrosive liquid. Finally, the application head may be any size or shape as required for the intended use and is not limited to being composed of just a sponge. The application head may be similar to a mop head or any type of wicking apparatus.

The materials used can vary according to need. For example, rigid PVC is used because of its strength, rigidity, durability and resistance to breakdown, even in the presence of many chemicals. Other materials are possible, including but not limited to metals and other types of plastics.

Additionally, other application heads are possible, such as cloth, natural, synthetic, or other materials that can transfer fluid from the reservoir to a targeted surface without spillage and with relatively precise control.

The shape and configuration of the components can also vary within the scope of the invention. For example, handle 12 does not have to be straight. The relative sizes of handle 12 and application head 30 can vary.

Another optional aspect of the invention utilizes device 10 in a kit that is sold together for the end user. The kit includes device 10 and at least a instruction sheet or card, preferably packaged together when presented for sale. The instruction sheet includes a mark or ruling of a pre-determined distance from one edge of the sheet or card, or a sticker or marker or instructions how to mark or indicate a certain height on a fluid-holding container (e.g. conventional plastic recyclable one gallon milk jug). The instruction sheet or card can include written indicia directing a user how to mix a solute (e.g. concentrated chemical) with water (or other solvent) to allow the user to prepare a desired amount of chemical for use with device 10. The mark or ruling on the instruction sheet or card, or the directions to apply a mark or indicia on the fluid-holding container, allow the user to have a handy and pre-determined gauge of how much concentrate to first put into the milk jug or other container. The instruction sheet or card instructs how much water to add to reach the correct level of dilution of the concentrate for application.

The kit could optionally contain instructions for different types or brands of chemicals, different concentrations, or other optional mixing configurations. The kit could optionally include the concentrate. It could optionally include a fluid-holding container for mixing the concentrate. Or the kit could optionally include such things as stickers, markers, or other indicia-created devices or items.

With some chemicals, e.g. herbicides, a kit of this type could allow the user to mix only so much as is needed at a certain time, and maintain the remainder in concentrate form. It can be substantially more economical. In one example, concentrate allows the chemical to be mixed for use at a cost of approximately one dollar per gallon, whereas buying the same chemical off-the-shelve, but pre-mixed and ready to use cost approximately nine dollars a gallon. By providing device 10 with instructions and tools as to how to mix concentrate can represent an advantageous and cost-effective combination.

What is claimed is:

1. An apparatus for selective application of liquid to a targeted surface comprising:
(a) an elongated handle;
(b) a fluid reservoir in the handle;
(c) an application head connected to the handle;
(d) a fluid control member operatively positioned between the reservoir and the application head.

2. The apparatus of claim 1 wherein the liquid is a chemical.

3. The apparatus of claim 2 wherein the liquid is selected from the set comprising a herbicide, an insecticide, a fertilizer, or combinations of one or more of same.

4. The apparatus of claim 3 wherein the liquid is an admixture of a concentrated solute and a solvent.

5. The apparatus of claim 1 wherein the targeted surface is a portion of a plant.

6. The apparatus of claim 1 wherein the handle is over three feet long and substantially hollow.

7. The apparatus of claim 1 wherein the handle comprises a plastic tube.

8. The apparatus of claim 7 further comprising a removable end cap for one end of the plastic tube.

9. The apparatus of claim 1 wherein the fluid reservoir comprises a space for containing at least several quarts of fluid.

10. The apparatus of claim 1 wherein the application head comprises a sponge.

11. The apparatus of claim 10 further comprising a plate having a first surface to which the sponge is attached, and a second surface mountable to the handle.

12. The apparatus of claim 1 wherein the control member comprises a valve with a manual on/off switch.

13. The apparatus of claim 1 wherein the control member is a part of the handle, and the sponge is attached to the control member.

14. A method of applying a liquid to a targeted surface comprising:
(a) storing the liquid in a portable, hand-carryable enclosed manner;
(b) transferring stored liquid to the targeted surface by capillary action by touching or swiping the targeted surface.

15. The method of claim 14 wherein the liquid is selected from the set comprising a herbicide, an insecticide, a fertilizer, or a combination of one or more of the same.

16. The method of claim 15 wherein the liquid is an admixture of a solute and a solvent.

17. The method of claim 14 wherein the targeted surface is a portion of a plant.

18. The method of claim 14 wherein the liquid is stored in an elongated tubular member.

19. The method of claim 14 wherein the capillary action as accomplished by providing a fluid pathway between the stored liquid and an application head.

20. The method of claim 19 wherein the application head comprises a sponge.

21. A kit useful for applying a liquid to a targeted area comprising:
(a) a handle defining an interior fluid reservoir and having an inlet with a removeable cap and an outlet;
(b) an application head comprising a sponge operatively connected to the handle so that the sponge is selectively in fluid communication with the fluid reservoir via a fluid pathway;
(c) a valve with a manually operable control operatively positioned in the fluid pathway;
(d) an instruction card including indicia comprising instructions and indicia for providing or facilitating a measuring mark on a separate fluid container for creating an admixture of solvent and concentrated solute.
